SEQUENCE LISTING (9 sequences)

SEQ ID NO: 1 - DNA, 24 bp, Artificial Sequence
  CHEMICAL SYNTHESIS OR IN VITRO ENZYMATIC SYNTHESIS
gatcctatatttttttaaatatag

SEQ ID NO: 2 - DNA, 35 bp, Artificial Sequence
  CHEMICAL SYNTHESIS OR IN VITRO ENZYMATIC SYNTHESIS
artcatctctctctctctggttccttccttccttc

SEQ ID NO: 3 - DNA, 30 bp, Artificial Sequence
  CHEMICAL SYNTHESIS OR IN VITRO ENZYMATIC SYNTHESIS
gaaggaaggaaccagagagagagagagaga

SEQ ID NO: 4 - DNA, 20 bp, Artificial Sequence
  CHEMICAL SYNTHESIS OR IN VITRO ENZYMATIC SYNTHESIS
gcgcgcttttgcgcgctctc

SEQ ID NO: 5 - DNA, 20 bp, Artificial Sequence
  CHEMICAL SYNTHESIS OR IN VITRO ENZYMATIC SYNTHESIS
cggccgttttcggccggaag

SEQ ID NO: 6 - DNA, 30 bp, Artificial Sequence
  CHEMICAL SYNTHESIS OR IN VITRO ENZYMATIC SYNTHESIS
atatgctctgcttttgcagagcatataagg

SEQ ID NO: 7 - DNA, 28 bp, Artificial Sequence
  CHEMICAL SYNTHESIS OR IN VITRO ENZYMATIC SYNTHESIS
tgaggtaggatttttcctacctcacctt

SEQ ID NO: 8 - DNA, 25 bp, Artificial Sequence
  CHEMICAL SYNTHESIS OR IN VITRO ENZYMATIC SYNTHESIS
gcagagcatataaggtgaggtagga

SEQ ID NO: 9 - DNA, 25 bp, Artificial Sequence
  CHEMICAL SYNTHESIS OR IN VITRO ENZYMATIC SYNTHESIS
tcctacctcaccttatatgctctgc

(19) United States
(12) Patent Application Publication
(10) Pub. No.: US 2003/0040613 A1
(43) Pub. Date: Feb. 27, 2003

(54) Structurally modified molecular decoys for the manipulation of cellular or viral replication and other uses relating thereto

(76) Inventors: Karl Duncan Bishop, Bangor, ME (US); Lee Ellen Bickerstaff, Bangor, ME (US)

Correspondence Address: Karl D. Bishop, 323 Husson Ave. Apt. 9, Bangor, ME, 04401, US

(21) Appl. No.: 09/841,236

(22) Filed: Apr. 24, 2001

Related U.S. Application Data
(60) Provisional application No. 60/199,162, filed on Apr. 24, 2000.

(51) Int. Cl. C07H 21/04; C07H 21/02

(52) U.S. Cl. 536/23.1

(57) ABSTRACT

Structurally modified, closed-ended nucleic acid decoys for the manipulation of cellular or viral replication processes are described. These decoys are resistant to cellular degradation, do not directly interact with host DNA and their structure does not require sophisticated knowledge of target DNA sequence. They have been demonstrated to be effective as transcription factor sequestration agents. By effectively competing for cellular transcription factors, these decoys will interfere with the normal transcription process, effectively turning off protein production and cellular replication. These decoys also have potential similar actions and effects in viruses. These decoys should have an impact in the treatment of cancer, AIDS, and other diseases.

### REFERENCES TO RELATED APPLICATIONS

This application is a non-provisional filing related to provisional patent application 60/199162, “Molecular Decoys for the Manipulation of Cellular Replication”, filing date Apr. 24, 2000, to Karl Bishop and Lee Bickerstaff.

### FIELD OF THE INVENTION

The present invention relates to novel nucleic acid motifs, structural modifications to these motifs, and the cellular effects and possible applications of these structurally modified nucleic acid decoys.

### BACKGROUND OF THE INVENTION

State-of-the-art treatment for many diseases and syndromes, including but not limited to cancers and AIDS, involves exposing both the invasive biologics and normal systemic cells to highly toxic compounds. Although effective, these treatments often have both low efficacy and highly detrimental side effects for the patient. It is clear that a more specified approach to the eradication of disease states is necessary. The current knowledge of cellular and viral genetics and replication allow for the development of a more directed therapeutic modality. By interfering with the processes that control cellular and viral replication, it should be possible to inhibit the replication of the biological entities that cause disease.

There has been great interest recently in the development of oligonucleotides as regulators of cellular nucleic acid biological function. Double-stranded DNA containing the genetic sequence for cellular control factors can be introduced into the system as a decoy, diverting control proteins from their endogenous DNA target. By diverting the control proteins from their endogenous target, the regulatory effects of such proteins can be altered. Double stranded DNA molecules containing such a target sequence can be prepared by chemically synthesizing a single stranded oligonucleotide containing the control sequence of interest, synthesizing the complement to this sequence, and allowing the two strands to anneal and hybridize. Introduction of such double stranded DNAs into whole cells, as a therapeutic regime, will be useful only if the construct is stable under the physiological conditions under which the cells remain viable. If the sequence length of the double stranded DNA being introduced is insufficient, the strands will tend to dissociate due to low binding stability (Cooney et al., 1988, Science 241:456). Additionally, segments of double stranded DNA have been shown to be susceptible to digestion by intercellular enzymes (nucleases) (Goodchild et al., 1988, Proc. Natl. Acad. Sci. USA 85:5507-5511). It is obviously necessary to develop double stranded oligonucleotides with the ability to effect cellular replication processes that are stable under physiological conditions.

Closed-ended nucleic acid decoys that seem to circumvent the problem of nuclease digestion have been developed. In U.S. Pat. No. 5,674,683 to Kool, Oct. 7, 1997 nuclease-resistant stem loop and circular oligonucleotides are described. These structures bind directly and irreversibly with all DNA in the host cell, regardless of whether the cell is invasive or native. While the structures described by Kool appear to efficiently deter the replication of invasive nucleic acid, they also appear to have similar inhibitory effects on host nucleic acids; the possible negative, long-term effects to the host are not discussed. While it is possible to theorize that the laws of thermodynamics would have these structures binding in an on/off competitive nature, there is no evidence to indicate that this is actually occurring. These same questions and limitations are also raised with respect to the oligonucleotides of U.S. Pat. No. 5,872,105 to Kool, Feb. 16, 1999 which describes single-stranded circular nucleic acid constructs that are nuclease resistant and bind directly and irreversibly with the host DNA. Additionally, these constructs contain nucleic acid sequence that is highly target-specific. The design of these constructs therefore limits their application, as they will interact only with their specific nucleic acid complement. The closed-ended constructs described in U.S. Pat. No. 5,683,985 to Chu et. al, Nov. 4, 1997 are also highly sequence-specific, which limits their application similarly. U.S. Pat. No. 4,777,129 to Dattagupta, et. al, Oct. 11, 1988 and U.S. Pat. No. 6,034,234 to Matsuo, et. al, Mar. 7, 2000 also discuss closed ended constructs specifically designed to have a clearly defined sequence reactive to a specific protein or antibody binding site. Although a great variety of constructs is defined and identified, all are dependent on the foreknowledge of the specific genetic sequence of any possible cellular replication control factors. While a great deal is currently known about cellular and viral replication, in reality we are still unable to halt the growth and replication of the majority of cancers and viruses. It is realistic to therefore theorize that these conditions may controlled by mechanisms other than the known, understood replication control factors that the constructs described in both patents are designed specifically against.

### SUMMARY OF THE INVENTION

Our invention describes structurally modified nucleic acid decoys that allow for the control of cellular or viral replication, are resistant to intracellular degradation, do not directly interact with host DNA, and do not require specific knowledge of the genetic sequences of any replication control factors. A nucleic acid decoy construct is envisioned for use in therapeutic regimes that could exhibit the efficacy of traditional protocols while minimizing the toxic side effects for the host. We have developed structurally modified, double stranded closed-ended nucleic acid decoys (dumbbells) to control cellular or viral replicative processes by functioning as sequestration agents. These decoys could become extremely useful in treating diseases by limiting the availability of enzymes and /or other factors that control the replication processes of cellular or viral structures. Our nucleic acid decoys are unique in that they contain a structural modification that creates a bend in the double stranded region of the decoy. This bend can be created by a variety of methods, including but not limited to bound metals and other ligands, abasic nucleic acids, base-pair mismatching, and modified nucleosides. Recent work has indicated that a commonly used chemotherapeutic agent, cisplatin, does not directly function by binding to tumor or host DNA, but rather by causing a bend in the DNA. This bent DNA structure has been shown to be attractive to a variety of intracellular replication control factors and proteins. By constructing a bent nucleic acid dumbbell, we have developed a nucleic acid decoy that is resistant to intracellular degradation and provides control of cellular replication processes without binding to host DNA. We envision that this invention could have significant impact in the treatment of cancer, AIDS, and other disease states.

### DESCRIPTION OF DRAWINGS

FIG. 1 schematically depicts DNA hairpins with overhanging ends, formed from self-complimentary DNA oligonucleotide sequence. Hairpin formation from oligonucleotides with as few as approximately 8 bases to a length in excess of 100 bases is theoretically possible.

FIG. 2 shows schematic representations of several DNA dumbbells. A variety of iterations of these structural formations can be developed. FIG. 2a depicts the double hairpin dumbbell.

FIG. 2b shows the schematic for the duplex hairpin dumbbell. FIG. 2c shows the schematic for the construction of the single-strand insert dumbbell.

FIG. 3A depicts a representation of cisplatin. FIG. 3B depicts a schematic representation of the structure of a cisplatin-dumbbell complex.

FIG. 4 shows a flow chart depicting transcription initiation in eukaryotes.

FIG. 5 shows a schematic structure and of the double hairpin dumbbell formed from SEQ. ID NO: 1, as described in the Example of the preferred embodiment.

FIG. 6 is a flow chart for the general method for the construction of DNA dumbbells.

FIG. 7 shows a schematic structure of the duplex hairpin dumbbell formed from SEQ. ID NO: 2, 3, 4, and 5, as described in the Example of the preferred embodiment.

### DESCRIPTION OF THE PREFERRED EMBODIMENT OF THE INVENTION

This invention is directed to the development of a structurally modified, double stranded, closed-ended nucleic acid decoy. This decoy is effective in the control of cellular replication process but does not require specific knowledge of the nucleic acid sequence of cellular replication factors to show efficacy. The decoy described in this invention also does not need to bind to the host DNA to perform its task effectively. The nucleic acid decoy is structurally modified to contain a bend, located within the double stranded region of the decoy. It is currently theorized that this bend attracts cellular replication factors that are necessary for the replication of biological entities, although I do not wish to be bound to this theory.

The current preferred embodiment of the invention, described in the example to follow, is a dumbbell DNA structure made from a self-complimentary nucleic acid sequence. Those skilled in the art can apply canonical methods to produce a dumbbell containing a double stranded central region, each end of which is capped with a single stranded region. Centrally located within this region is the binding site for the structure modifying agent. Binding of dumbbell and structure modifying agent is accomplished under conditions specific to the agent being employed to accomplish structural modification. The purified, structurally modified dumbbell can then be incubated with a variety of cellular extracts, purified transcription factors or other proteins and assayed for efficacy as a sequestration agent for these compounds. In the example for the preferred embodiment described below, the structurally modified dumbbell can be shown to effectively sequester a human transcription factor even though the dumbbell does not contain the sequence of the known binding site for this protein.

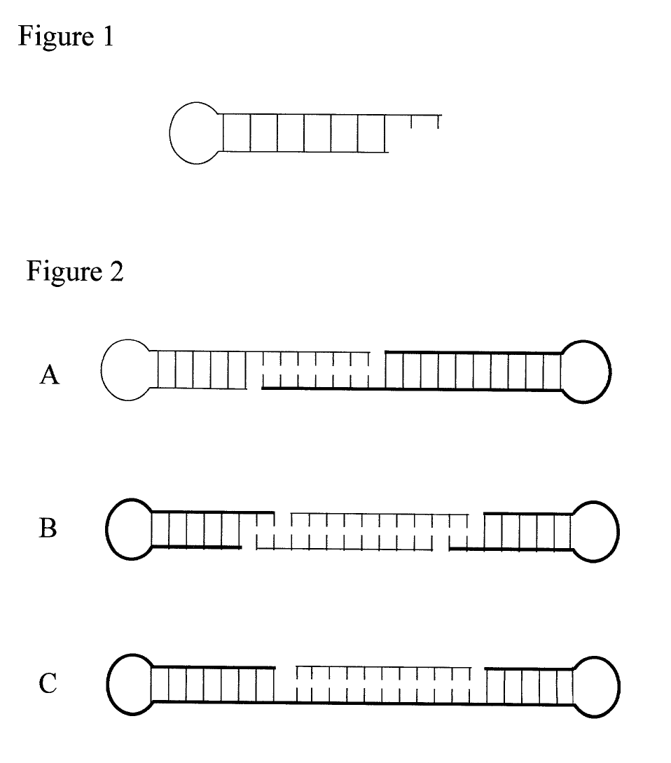

The dumbbells used in the preferred embodiment are constructed from self-complimentary oligonucleotide sequence. This oligonucleotide is heated to near boiling (of water) for approximately 10 minutes, then plunged immediately into an ice bath for about an equal time length. This rapid temperature change causes the oligonucleotide sequence to flip around on itself, with the thymine stretch as the centerpoint. Base-pairing of complimentary nucleic acids occurs, forming what is known as a DNA hairpin. FIG. 1 shows a representative, schematic DNA hairpin. The hairpins in this study were designed to have complimentary, overhanging ends. When incubated under the appropriate conditions, these ends will form base-pairs and anneal. Incubating the double hairpin structure with T4 DNA ligase will ligate the overhanging ends, yielding a stable, nuclease resistant dumbbell. FIG. 2A shows a schematic dumbbell. Although this is the canonical method for making dumbbells and is used in the preferred embodiment of the invention, several other methods have been envisioned and/or employed. FIGS. 2B, 2C, and 2D depict schematics for some of these, including but not limited to the duplex-hairpin dumbbell and the single-stranded insert dumbbell. While generally following the canonical method for dumbbell construction (i.e., high heating, rapid chilling, ligation of either blunt or sticky ends), both methods allow for the introduction of specific structural-modifying sites into the dumbbell. The specific length of the dumbbell, the oligonucleotide sequence used, and the exact number and location of structural modifying sites to be introduced is dependent on the study being undertaken.

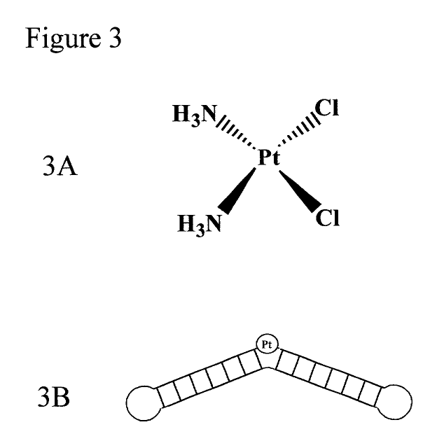

FIG. 3A shows a structural representation for cis-diamminedichloroplatinum II (cisplatin), the agent employed in the preferred embodiment of the invention to cause bending of the DNA dumbbell. Cisplatin chemistry shows that the binding region of the molecule has a strong preference for -GG- nucleic acid moieties, causing a bend in the target nucleic acid sequence. By incorporating a -GG- moiety in the current embodiment, cisplatin will bind to the dumbbell and cause it to bend. FIG. 3B shows a schematic for the cisplatin bent dumbbell. Recent studies have indicated that it is this bend in the nucleic acid structure that bestows upon cisplatin much of its efficacy in the treatment of disease. This enhanced efficacy is due to the recognition and binding of cellular proteins to the cisplatin-DNA intrastrand adduct. It has been seen that these interactions can interfere with transcription by sequestering essential transcription factors from their native binding sites. The bent dumbbell structure therefore effectively acts as a sequestration agent for proteins essential to cellular replication. It should be obvious to anyone practiced in the art that a variety of agents, including but not limited to pharmaceuticals, metals, nucleic acid analogs and variation of base-pair matching and number, could all cause a bend in the DNA.

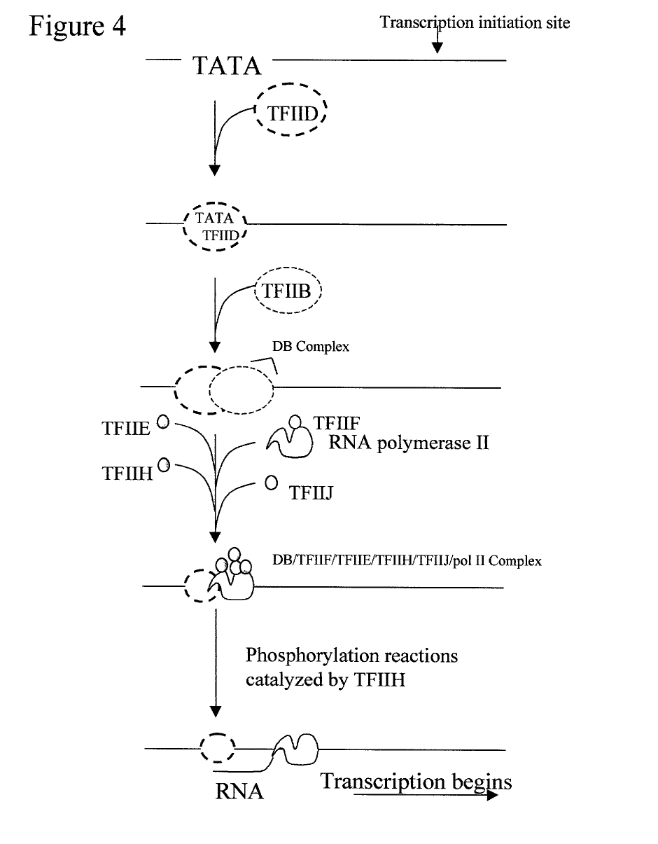

In 1958, Francis Crick enunciated the “central dogma of molecular biology”. This scheme outlined the residue-by-residue transfer of biological information as encoded in the primary structure of the informational biopolymers, nucleic acids and proteins. The predominant path of information transfer, DNA→RNA→protein, postulated that RNA was an information carrier between DNA and proteins, the agents of biological function. In 1961, Francois Jacob and Jacques Monod extended this hypothesis to predict the properties of the RNA intermediate, which became known as messenger RNA (mRNA). Since Jacob and Monad's 1961 hypothesis, it has been determined that cells contain three different major classes of RNA, all of which participate in protein synthesis. All of these RNAs are synthesized from DNA templates by DNA-dependent RNA polymerases in the process known as transcription. However, only mRNAs direct the synthesis of proteins. Protein synthesis occurs via the process of translation, wherein the instructions encoded in the sequence of bases in mRNA are translated into a specific amino acid sequence. Transcription is tightly regulated in all cells. In a differentiated eukaryotic cell, only about 0.01% of the genes are undergoing transcription at any given time. Such differentiated cells express only the information needed for their biological function, not the full genetic potential encoded in their chromosomes. Eukaryotic cells have three classes of RNA polymerase, each of which synthesizes a different class of RNA. RNA polymerase II (RNA pol II) transcribes protein-encoding genes, and thus is responsible for the regulated synthesis of mRNA, the RNA responsible for the direction of protein synthesis. RNA pol II interacts with its promoters via transcription factors. Transcription factors are DNA-binding proteins that recognize and accurately initiate transcription at a specific promotor sequence. RNA pol II promotors commonly consist of two separate sequence features, the core element, where general transcription factors bind, and regulatory elements known as enhancers or silencers. The core region often consists of a TATA box (a TATAAA consensus element) and the transcription start site. An important role of the TATA box is to indicate the site of the initiator element, where transcription is initiated. A universal set of proteins, the basal apparatus, binds the core promotor and initiates transcription. This basal apparatus consists of RNA pol II and the general transcription factors TFIIB, TFIID, TFIIE, TFIIF, and TFIIH. TFIID consists of a TATA-binding protein (TBP) which directly recognizes the TATA box, and a set of TBP-associated factors (TAFs), which have positive or negative effects on transcription. Although several models of transcription initiation exist, it is generally agreed that the TFIID/TBP complex binds in the minor groove of DNA. This binding opens the TATA sequence, and other components of the TFIID heteronomer (i.e., the TAFs) sit on TBP. All known eukaryotic genes(including those lacking a TATA box and those transcribed by RNA polymerases other then RNA pol II) rely on TBP. A preinitiation complex forms at the TATA-containing promotor. Binding of TFIID, TBP, and other polypeptides is stimulated by TFIIA. TFIID bound to the TATA motif recruits TFIIB, forming a TFIID/TFIIB complex (DB complex). In association with TFIIF, RNA pol IIA joins the DB complex to give the DBpolF complex. TFIIE and TFIIH then associate with the DBpolF complex, yielding the preinitiation complex. Melting of the DNA duplex around the initiator element (located within the DNA sequence) generates an open complex and transcription of the DNA ensues. This transcription yields the MRNA necessary that will eventually be translated into the protein encoded for by the DNA undergoing transcription. FIG. 4 shows a schematic flow chart for this process.

The gel mobility shift assay is a simple and rapid method that has been widely used in the study of sequence-specific DNA-binding proteins, such as transcription factors. In a gel matrix, protein/DNA complexes migrate slower than either free protein or free DNA. The gel mobility shift assay takes advantage of this property, making it simple to distinguish between a protein/DNA complex and the free forms of either component. In this competitive assay, the dumbbells described above will compete for binding to TFIID protein with duplex DNA that contains the known sequence for the TFIID binding site. Because the duplex DNA containing the binding site is radioactively labeled, a shift in its mobility on a polyacrylamide gel will be noted. When the binding site sequence is bound to the TFIID, it will be part of the larger complex and its mobility through the gel will be retarded. It will migrate only into the upper portion of the gel, reflecting the apparent molecular weight of the protein/binding site complex. When the dumbbell structures bind to the TFIID protein, the labeled binding sequence duplex will appear as a rapidly moving band, located near the bottom of the gel where its molecular weight is accurately represented.

### EXAMPLE

The preferred embodiment for this experiment is to form a double stranded DNA dumbbell with single stranded connecting poly-thymine loops from simplistic DNA hairpins. Self-complimentary 5′phosphorylated DNA oligonucleotides with a length of 24 bases were purchased from the H.H.M.I./Keck Oligonucleotide Synthesis Laboratory, Yale University, New Haven, Conn., where they were produced by canonical methods using solid phase phosphoramidite chemistry. The sequence of these oligonucleotides is

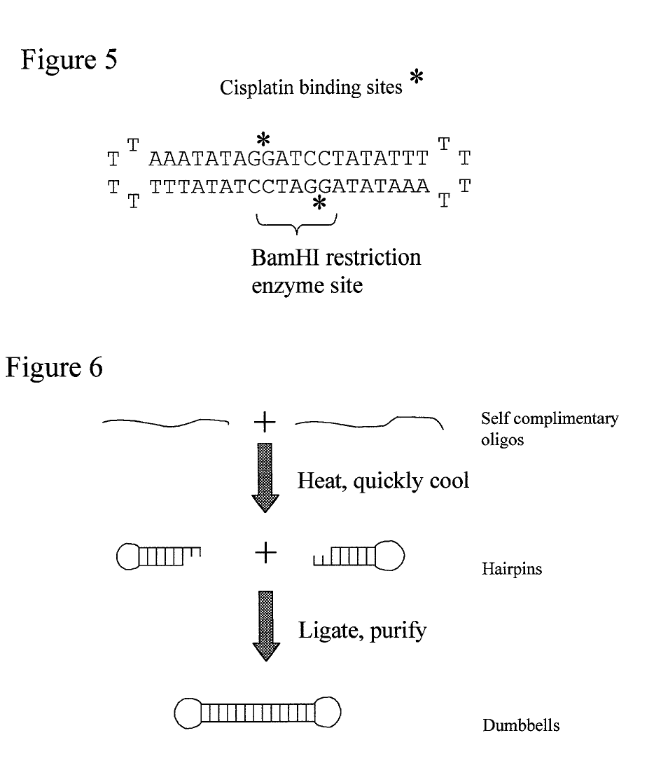

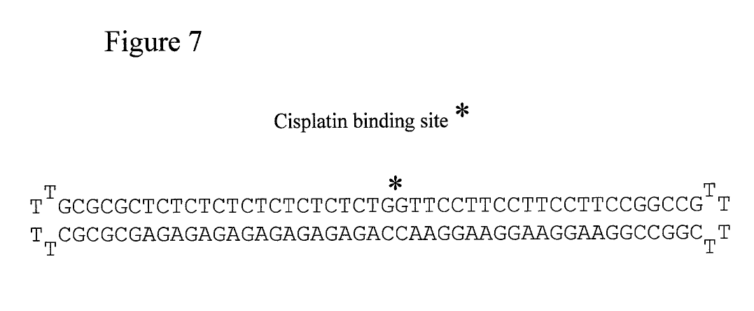

5′-GATCCTATATTTTTTTAAATATAG-3′ (SEQ ID NO:1). The DNA dumbbell resulting from this sequence will have the following characteristics: (a) The double stranded DNA sequence is connected by single stranded poly-thymine loops; (b) The resulting dumbbell will have a centrally located -GG- moiety, which is the preferred binding site for cisplatin; and (c) The central portion of the dumbbell containing the platinum binding site, will also contain a site which is sensitive to the restriction endonuclease BamHI, which is useful in the purification of the platinated dumbbell structure. FIG. 5 shows the this dumbbell, with cisplatin binding sites and BamHI restriction site indicated. To create hairpins, 10 ug DNA (vol. 1 ul) was combined in a microfuge tube with 1 ul 10×T4 DNA ligase buffer and 7 ul distilled, deionized water. The sample was heated at 95° C. for ten minutes and then plunged into ice and allowed to incubate for an additional ten minutes. One ul T4 DNA ligase was subsequently added and the sample incubated for 16 hours at 16° C. The sample was heated at 80° C. for 10 minutes to inactivate the ligase buffer. The resultant dumbbell sample was then passed through a centrifugal size-exclusion chromatography column, from which it eluted in water. FIG. 6 shows a schematic flow chart for this process. After retaining an aliquot for a control sample, the remaining dumbbell was incubated with cisplatin in a 1.2:1 molar ratio of platinum to DNA. The sample was incubated for 96 hours at ambient temperature in the dark. At the end of the incubation, the sample was passed over a centrifugal size-exclusion chromatography column to remove unbound platinum and elute the platinated dumbbell into water. The platinated dumbbell sample was then incubated with 1/10 volume 10×BamHI buffer and Bam HI at a concentration of 5 units enzyme/ug DNA. The restriction recognition site for BamHI is -GGATCC-, with cutting occurring between the -GG- residues. Previous experiments have shown that when the -GG- site is blocked by platinum binding, the enzyme cannot cleave the DNA. This therefore becomes a handy way to distinguish platinum-bound dumbbell from unbound; the restriction endonuclease cleaves the unplatinated dumbbell into two DNA hairpins. Passing the digested sample through a centrifugal size-exclusion chromatography column allows for the purified, platinated dumbbell to be eluted in water. The sample is then dried via a centrifugal vacuum system and frozen for further use.

An alternate strategy for producing structurally modified nucleic acid decoys is to produce a platinated duplex-hairpin dumbbell. This type of dumbbell is formed by allowing two complimentary single stranded oligonucleotides with dissimilar three prime or five prime ends (SEQ ID NO:2) (SEQ ID NO:3), one strand of which contains a centrally-located ligand binding site, to anneal and form a double stranded linear DNA duplex structure with overhanging sticky ends. This duplex is then ligated with hairpins complimentary to the overhanging sticky ends of the duplex which are similar in structure and construction to those listed above but without ligand binding site or restriction endonuclease cleavage site (SEQ ID NO:4) (SEQ ID NO:5). A digestion step employing Exonuclease III, which cleaves DNA where a gap in the linear DNA backbone is detected, significantly reduces the size of non-intact dumbbells. A final purification using a double chambered centrifugation dialysis unit having a membrane with an appropriate molecular weight cut-off yields purified, structurally modified nucleic acid decoy in the retentate. The more complicated design methodology of this dumbbell structure allows for ligand binding as an initial step in the construction process. The ligand is bound to the single stranded oligonucleotide, which can then be purified by HPLC, to insure that only structurally modified dumbbells will be produced. An additional benefit realized when using this method is the ability to ensure the production of a dumbbell with a single structure-modifying site. The construction process then proceeds as outlined above.

Three dumbbell structures were made following the above protocol to be used in a competitive assay to determine if structurally modified DNA constructs could effectively compete for cellular transcription factors, which could lead to sequestration of these factors, thereby affecting cellular replication. To include positive and negative controls, the following dumbbells were constructed and tested: The consensus dumbbell, formed from dissimilar haipins with complementary overhanging ends and containing the canonical binding site for the transcription factor being tested (SEQ ID NO:6) (SEQ ID NO:7); the experimental dumbbell constructed via the duplex-hairpin strategy, with sequence containing the ligand binding site and ligand(SEQ ID NO:2) (SEQ ID NO:3) (SEQ ID NO:4) (SEQ ID NO:5); and a control dumbbell, which was of the same sequence as the experimental sample but had no ligand bound to its binding site. The ligand used in this study was cis-diamminedichloro-platinum(II), more commonly referred to as cis-DDP or cisplatin. These constructs were analyzed for their ability to compete for binding to the transcription factor TFIID. TFIID is a general transcription factor exhibiting specific DNA-binding to the TATA box. For many genes, TFIID is necessary, and in conjunction with RNA polymerase II sufficient, to initiate basal transcription. Construct competition for the TFIID binding site is anticipated to therefore effect transcription of genetic material. The binding characteristics of the three dumbbells described above were evaluated using a gel mobility shift assay.

To conduct the competitive binding assay, a double stranded linear oligonucleotide containing the known recognition sequence for the TFIID binding site (consensus duplex) (SEQ ID NO:8) was radiolabeled with 32P-ATP using T4 polynucleotide kinase according to standard procedures. This consensus duplex was then incubated with an equimolar amount of TFIID protein. After a 15 minute incubation on ice, varying concentrations (3.5, 0.35, 0.035, and 0.0035 pM) of the dumbbells under study (control, experimental, and consensus) were added to the TFIID/consensus duplex reactions in an attempt to compete the TFIID protein from its consensus duplex. Samples were incubated on ice for another 30 minutes and then separated on a 5% polyacrylamide gel. After electrophoresis, the gel was subjected to autoradiography for varying times until acceptable autoradiographs were obtained.

The autoradiographs clearly show that all three of the dumbbell structures competitively bind with the TFIID binding site (data not shown). Dumbbell concentrations of 3.5 and 0.35 picomolar successfully compete with the consensus duplex for binding TFIID, as indicated by a lack of radiolabeled band at the expected, higher molecular weight site. The unlabelled dumbbell has occupied the TFIID binding site, and the consensus duplex can be observed at the bottom of the autoradiogram. At lower dumbbell concentrations (0.035 and 0.0035 pM), consensus duplex/TFIID binding occurs and can be observed as the higher molecular weight band.

### CONCLUSION, RAMIFICATION AND SCOPE OF THE INVENTION

These results demonstrate the ability of structurally modified nucleic acid decoys to successfully compete for binding of transcription factor control sites. The decoys of the invention do not require sophisticated knowledge of genetic sequence and do not bind to host DNA, yet they appear to effectively sequester cellular control mechanisms, thereby allowing for the manipulation of cellular or viral replication.

While the above description contains many specificities, these should not be construed as limitations on the scope of the invention, but rather as an exemplification of one preferred body thereof. Many other variations are possible. For example, it is possible that these same principles could be applied to effect the activity of HIV integrase and its role in the replication of human immunodeficiency virus. Other cellular or viral replication schemes could possibly be effected by similar but specific constructs. Accordingly, the scope of the invention should be determined not by the embodiment illustrated, but by the appended claims and their legal equivalents.

We claim:

1. A continuous circular oligonucleotide structure comprising:
(a) a double stranded central region,
(b) a single stranded means for connecting the opposing ends of said double stranded region,
(c) one or more structure modifying sites.

2. The continuous circular oligonucleotide structure of claim 1 wherein said double stranded region is composed of deoxyribonucleotides or ribonucleotides.

3. The continuous circular oligonucleotide structure of claim 2 wherein said double stranded region composed of said deoxyribonucleotides or ribonucleotides may include a limited number of non-nucleotide moieties.

4. The continuous circular oligonucleotide structure of claim 1 wherein said single stranded means for connecting opposing ends of said double stranded region comprising deoxyribonucleotides or ribonucleotides or non-nucleotide moieties or any combination thereof.

5. The continuous circular oligonucleotide structure of claim 1 wherein said structural modifying site is located within said double stranded region.

6. The continuous circular oligonucleotide structure of claim 5 wherein said structural modifying site provides a means for ligand binding.

7. The continuous circular oligonucleotide structure of claim 5 wherein said structural modifying site is comprised of both deoxyribonucleotides and ribonucleotides.

8. The continuous circular oligonucleotide structure of claim 5 wherein said structural modifying site is comprised of nucleotides which do not exhibit Watson-Crick hydrogen bonding.

9. The continuous circular oligonucleotide structure of claim 5 wherein said structural modifying site provides a means for chemical intercalation.

10. The continuous circular oligonucleotide structure of claim 5 wherein said structural modifying site is comprised of non-nucleotide moieties.

11. The continuous circular oligonucleotide structure of claim 5 wherein said structural modifying site is comprised of modified nucleosides.

12. The continuous circular oligonucleotide structure of claim 5 wherein said structural modifying site is comprised of modified nucleotides.
